(19) United States
(12) Patent Application Publication
(10) Pub. No.: US 2008/0207896 A1
(43) Pub. Date: Aug. 28, 2008

(54) Process For the Manufacture of Mirtazapine

(75) Inventor: Carmen Arnalot Aguilar, Barcelona (ES)

Correspondence Address: LADAS & PARRY LLP, 26 WEST 61ST STREET, NEW YORK, NY, 10023, US

(73) Assignee: MEDICHEM, S.A., Sant Joan Despi (ES)

(21) Appl. No.: 11/630,505

(22) PCT Filed: Jul. 19, 2005
(86) PCT No.: PCT/EP2005/053493

§ 371 Date: Dec. 28, 2007

(51) Int. Cl. C07D 471/14

(52) U.S. Cl. 540/578

(57) ABSTRACT

Improved process for manufacturing mirtazapine. A process is described for preparing mirtazapine starting from a compound of formula (II), which is subjected to a ring cyclization, obtaining mirtazapine for pharmaceutical use in crystalline and anhydrous form.

### FIELD OF THE INVENTION

The present invention relates to a process for preparing mirtazapine.

### BACKGROUND OF THE INVENTION

Mirtazapine, 1,2,3,4,10,14b-hexahydro-2-methyl-pyrazi no[2, 1-a]pyrido[2,3-c][2]-benzazepine, having the formula (I):

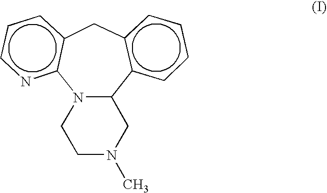

is approved, under the trademark Remeron®, by the U.S. Food and Drug Administration, for the treatment of depression.

Mirtazapine may be made by methods described in U.S. Pat. No. 4,062,848 (Akzona Incorporated). Example 1 of U.S. Pat. No. 4,062,848 explicitly discloses a process for preparing mirtazapine by adding concentrated sulfuric acid to 1-(3-hydroxymethylpyridyl-2)-2-phenyl-4-methylpiperazine of formula (II) at room temperature.

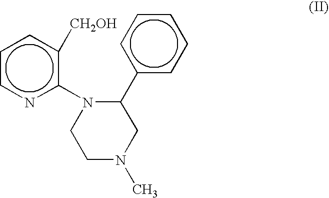

However, there are some defects in this process so that the process is not satisfactory for industrial implementation. Some of the defects are the following:

Concentrated sulfuric acid is added dropwise at room temperature to a solid compound so the stirring of the mixture is not efficient and the reaction control is difficult.

The reaction mixture is extracted with chloroform so impurities are also extracted.

Mirtazapine is crystallized by addition of ether which is very difficult to handle in large scale production.

Mirtazapine is recrystallized from petroleum ether 40-60 which is very difficult to handle in large scale production.

In Examples 2 and 3 of WO 00/62782 it is disclosed that the same reaction can be carried out by adding 1-(3-hydroxymethylpyridyl-2)-2-phenyl-4-methylpiperazine of formula (II) to concentrated sulfuric acid. In Example 2 the reaction is carried out at room temperature for 4 hours followed by heating for one hour to about 500 to 60° C. In Example 3 the reaction is carried out at 35° C. for 6 hours.

In Preparation Example 1 and Preparation Example 2 of EP 1 209 159 A2 it is disclosed that the compound of formula (II) is added to the concentrated sulfuric acid in divided portions at 5° to 30° C. and that the mixture is stirred at 30° to 40° C. for 8 hours. In addition, mirtazapine is obtained from a mixture of toluene and heptane.

According to the “Detailed Description of the Preferred Embodiments” of patent application EP 1 209 159 A2:

It is desired that the temperature of the concentrated sulfuric acid (when the pyridinemethanol compound is added to the concentrated sulfuric acid) is 0° to 40° C., preferably 50 to 35° C., in order to suppress heat generation and generation of tarred impurities. (See [0017] on column 3).

In the case where the pyridinemethanol compound is added to the sulfuric acid, it is preferred that the pyridinemethanol compound is added in divided portions to the concentrated sulfuric acid, in order to cause the reaction to proceed efficiently. For instance, it is preferred that the pyridinemethanol compound is added to the concentrate sulfuric acid in 5 to 20 divided portions. (See [0018] on column 3).

It is important to take into account that the pyridinemethanol compound is added as a solid to the sulfuric acid, which is a disadvantage from an industrial point of view.

The known processes have various drawbacks in that they are complicated, require the observation of particular reaction conditions and do not always lead to mirtazapine of sufficient quality for pharmaceutical use. Accordingly, there is a need for a process useful for the preparation of mirtazapine in high quality on an industrial scale.

### SUMMARY OF THE INVENTION

It is an object of the present invention to provide a process for the preparation of mirtazapine which avoids the drawbacks of prior art processes. Further it is an object of the invention to provide a process by which mirtazapine in high purity is obtained.

According to the invention, there is provided a process for preparing mirtazapine comprising the steps of

(a) dissolving or suspending a compound of the formula (II)

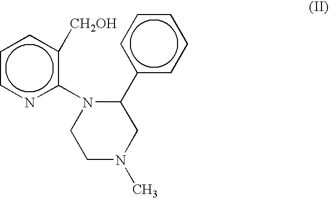

in a liquid diluent to obtain a first mixture;

(b) adding a ring closing reagent to the first mixture to obtain a second mixture;

(c) allowing the formation of mirtazapine in the second mixture;

(d) isolating mirtazapine from the second mixture.

One of the preferred embodiments of the invention relates to a process for preparing mirtazapine which comprises purifying of mirtazapine by crystallization from an organic ester solvent.

### DETAILED DESCRIPTION OF THE INVENTION

The present invention relates to an improved process of making anhydrous mirtazapine from compound of formula (II).

The present invention also provides a new method for making pure anhydrous mirtazapine by purifying crude anhydrous mirtazapine by recrystallization from ethyl acetate.

In the process of the present invention, the compound of formula (II) is dissolved or suspended in a liquid diluent. A ring closing reagent is added to the resulting mixture and the reaction is carried out at a selected temperature. The completion of the reaction may be monitored by HPLC (High performance liquid chromatography) or thin layer chromatography.

The time needed for the completion of the ring closure varies with the temperature of the reaction. Higher reaction temperatures generally require shorter reaction times, while lower reaction temperatures generally require longer reaction times.

Diluents that may be used are water, halogenated hydrocarbons such as dichloromethane, hydrocarbons such as toluene or anisol. Diluents that are preferred are dichloromethane and water, particularly water. The amount of the diluent is 0.25 to 5 parts by weight, more preferably 0.4 to 1.1 parts by weight based on 1 part by weight of the compound of formula (II).

Suitable ring closing reagents are dehydrating agents. Dehydrating agents that may be added to the reaction mixture include acids and acid derivatives, such as sulfuric acid, concentrated sulfuric acid, phosphoric acid, phosphorous oxychloride. The dehydrating agents that are particularly preferred are sulfuric acid and concentrated sulfuric acid. The concentration of the concentrated sulfuric acid is preferably in the range of 96 to 99 wt %. If the ring closing reagent can react with the diluent, an excess of ring closing reagent sufficient to allow the formation of mirtazapine is used. The preferable amount of the ring closing reagent is 1 to 6 parts by weight, more preferably 2.5 to 5.0 parts by weight based on 1 part by weight of the compound of formula (II).

It is particularly preferred to use water as a diluent and concentrated sulfuric acid as a ring closing reagent. The particularly preferred amount of diluent is then 0.4 to 0.6 parts by weight of the compound of the formula (II), and the particularly preferred amount of ring closing reagent is 4 to 5 parts by weight of the compound of the formula (II).

The ring closing reagent is preferably added as a thin stream to a mixture of compound (II) and diluent at a rate that keeps the temperature of the reaction mixture below its reflux temperature. After the addition of the ring closing reagent, it is preferable that the mixture is stirred at a temperature of about room temperature to reflux temperature for about 1 to 24 hours. A temperature of below 90° C., in particular below 80° C., and a reaction time of 1 to 3 hours, in particular about 2 hours, are preferred. These conditions are especially preferred if water is used as a diluent and concentrated sulfuric acid is used as a ring closing reagent.

When the reaction has taken place, the ring closing reagent is diluted or destroyed, for instance, by the addition of a thin stream of the reaction mixture to water or to an aqueous alkali solution. It is preferred that the temperature of the reaction mixture during the addition is from 0° to 30° C. When the ring closing reagent is diluted or destroyed with water, then the mixture is made alkaline by the addition of an aqueous alkali solution. It is preferred that during basification there is present a non-water miscible solvent in order to keep mirtazapine dissolved all time. Any alkali can be used, including but not limited to, sodium hydroxide, potassium hydroxide, sodium carbonate, sodium bicarbonate and ammonium hydroxide. Among them, sodium hydroxide and ammonium hydroxide are preferred.

In a preferred embodiment of the process, the reaction mixture is added to water and a non-water miscible solvent is added before basification. In another preferred embodiment, the reaction mixture is added to an aqueous alkali solution. In this case, the non-water miscible solvent has previously been added to the aqueous alkali solution or to the reaction mixture. Furthermore, it is possible to add water or an aqueous alkali solution to the reaction mixture. Also in this case it is preferred that a non-water miscible solvent is present during the contact of the reaction mixture and any basic component.

Common non-water miscible solvents such as toluene or dichloromethane can be used. Toluene is preferred. During the extraction of crude mirtazapine, the pH of the aqueous alkaline layer is adjusted, if necessary, so that the pH becomes not less than 8, becomes 8 to 10, preferably 8.5 to 9.5.

The isolated non-water miscible layer containing mirtazapine can be treated with a desiccant to remove moisture therefrom, if desired. The desiccant can be any conventional desiccant, including but not limited to, anhydrous sodium sulfate, anhydrous magnesium sulfate and molecular sieves. Alteratively, azeotropic distillation can be used to remove moisture.

In addition, a decolorizing agent is also preferably added to the non-water miscible layer containing mirtazapine, in order to improve the quality attributes like colour and purity of the resulting anhydrous mirtazapine crystals. The decolorizing agent can be any conventional decolorizing agent, including but not limited to, alumina, activated alumina, silica and charcoal. The decolorization temperature is preferably between room temperature and 80° C., more preferably below 40° C.

Crude anhydrous mirtazapine is obtained by removing the non-water miscible solvent by distillation and adding a different solvent which allows the formation of crystalline anhydrous mirtazapine.

The distillation of the non-water miscible solvent can be carried out by any distillation means, preferably by distillation under reduced pressure. The reduced pressure is such that the temperature during distillation is below 50° C., more preferably below 40° C. Solvent is added to the residue of distillation and the distillation under reduced pressure is continued. Then solvent is added to the residue and the mixture is heated to a suitable temperature. Suitable temperatures include, for example, the reflux temperature of the solvent. Crude anhydrous mirtazapine precipitates upon cooling of the reaction mixture, preferably to −10° to 10° C., more preferably to 0° to 5° C. After cooling, the mixture is stirred at 0° to 5° C. for at least 1 hour and for up to about 6 hours, more preferably for up to about 4 hours to increase the yield of the anhydrous mirtazapine crystal. The anhydrous crude mirtazapine crystals can preferably be collected by filtration or centrifugation.

The wet crude anhydrous mirtazapine crystals are preferably recrystallized again. The crude anhydrous mirtazapine is mixed with fresh solvent and heated to reflux temperature. The amount of solvent is the necessary amount to obtain a solution at reflux temperature. The solution is cooled to 0° to 5° C. and stirred at this temperature for about 6 hours, more preferably for about 4 hours, to increase the yield of the anhydrous mirtazapine crystal. The anhydrous mirtazapine crystals can preferably be collected by filtration or centrifugation. The collected anhydrous mirtazapine crystals are dried, preferably under reduced pressure, to reduce the residual solvent in the anhydrous mirtazapine crystals. The drying temperature is preferably 20° to 70° C., more preferably 40° C. to 60° C. More preferably the reduced pressure is about 100 mm Hg, and the product is dried at about 40° C. for about 2 hours, followed by about 4 hours at about 60° C. to remove the residual solvent from the anhydrous mirtazapine crystals.

The solvent which allows the formation of crystals of anhydrous mirtazapine is an organic ester, preferably an organic acetate, more preferably ethyl acetate.

The starting compound of formula (II) is commercially available. Alternatively, compound (II) can be prepared following the methods described in U.S. Pat. No. 4,062,848 and purified by recrystallization from ethyl acetate, if necessary.

The following examples are given for the purpose of illustrating the present invention and shall not be construed as being limitations on the scope or spirit of the invention.

### EXAMPLE 1

This example shows the preparation of mirtazapine using 1 part compound of formula (II)+0.5 parts water+4.6 parts dehydrating agent (parts are in weight) and keeping the reaction mixture for 2 h below 80° C.

In a suitable reactor are loaded:

3.5 kg of deionized water

7.0 kg of 1-(3-hydroxymethylpyridin-2-yl)-4-methyl-2-phenylpiperazine (i.e. the compound of formula (II)).

The reactor is cooled down to 10° C. and with continuous stirring. 32.2 kg of sulfuric acid (96.10 wt %, corresponding to 30.94 kg H₂SO₄) are added maintaining the temperature below 80° C. After the addition, the reaction mixture is maintained at 75-80° C. during 2 hours. Then the reactor content is cooled down to room temperature and added to 40 kg of deionized water (previously cooled at not more than 15° C.) keeping the temperature below 25° C.

Mirtazapine is then extracted by addition of 57 kg of toluene and 54 kg of 26% ammonium hydroxide to adjust the pH to 8.9-9.3. The phases are separated and the aqueous phase is re-extracted with 13 kg of toluene.

The phases are separated and the aqueous phase is re-extracted with 8 kg of toluene.

The organic phases are loaded into a suitable reactor and washed with 61 kg of deionized water.

The organic extracts are treated with anhydrous sodium sulfate and filtered. Then the solution is 2 times de-coloured with active charcoal and filtered.

Toluene is distilled off under vacuum without exceeding 40° C. and mirtazapine is crystallized from ethyl acetate and filtered.

The wet solid obtained is re-crystallized from ethyl acetate, filtered and dried at 40° C. and then at 60° C. at a pressure of not more than about 100 mm of Hg. 4.7 Kg. (Molar yield: 72%) of anhydrous crystalline mirtazapine are obtained.

The solid is then milled, sieved through a 500 μm screen and blended for at least 2 hours.

The HPLC purity is 99.7%. Residual solvents (as determined by gas chromatography): toluene below detection limit of 100 ppm, ethyl acetate 299 ppm.

### EXAMPLE 2

This example shows the preparation of mirtazapine using 1 part compound of formula (II)+0.5 parts water+4.6 parts dehydrating agent (parts are in weight) and keeping the reaction mixture for 2.5 h at 40° C.+1 h at 60° C.+1 h at 80° C.

To a suspension of 20 g of 1-(3-hydroxymethylpyridin-2-yl)-4-methyl-2-phenylpiperazine in 10 ml of deionized water, 50 ml of sulfuric acid (96.10 wt %) is added dropwise at 24 to 34° C. during 15 minutes. Then, the solution is heated to 40° C. and maintained for 2 hours and 30 minutes, after to 60° C. and maintained 1 hour and finally to 80° C. and maintained 1 hour. As a result, a solution is obtained.

At room temperature, 50 ml of water are slowly added. Next 188 ml of toluene are added and then the pH of the mixture is adjusted to about 9 with 28% ammonium hydroxide. The temperature during the pH adjustment is maintained below 30° C.

This solution is allowed to separate into two layers and the aqueous layer is extracted again with toluene.

Then the organic phases are joined and washed with 175 ml of water. Thereafter the toluene is dried with sodium sulfate and decolorized with 0.6 9 of charcoal. After 45 minutes, the solution is filtered. The solvent is evaporated under reduced pressure to dryness. The HPLC purity is 99.45%.

21 ml of ethyl acetate is added to the residue, heated to reflux temperature and then cooled to 5° C. and stirred at the same temperature for one hour and filtered.

The resulting crystals are washed with 2 ml of ethyl acetate to give wet crude anhydrous mirtazapine. The HPLC purity is 99.94%.

The crude mirtazapine is crystallized with 15 ml of ethyl acetate. Thereafter the mixture is cooled to 0-5° C. and stirred at the same temperature for 1 hour.

After filtering and washing with 2 ml of ethyl acetate, the wet crystals are dried under reduced pressure at 60° C. until constant weight, to give purified anhydrous mirtazapine crystals. The HPLC purity is 99.98%. Residual solvents (as determined by gas chromatography): toluene below detection limit of 100 ppm, ethyl acetate 175 ppm.

### EXAMPLE 3

This example shows the preparation of mirtazapine using 1 part compound of formula (II)+0.5 parts water+4.6 parts dehydrating agent (parts are in weight) and keeping the reaction mixture for 7 h at 60° C.

To a suspension of 20 g of 1-(3-hydroxymethylpyridin-2-yl)-4-methyl-2-phenylpiperazine in 10 ml of deionized water, 50 ml of sulfuric acid (96.10 wt %) is added dropwise at 20 to 60° C. during 20 minutes. Then the solution is heated to 60° C. and maintained for 7 hours.

As a result, a solution is obtained which is cooled to 0-5° C. and 50 ml of water are slowly added. Next 188 ml of toluene are added and then the pH of the mixture is adjusted to about 9 with 28% ammonium hydroxide. The temperature during the pH adjustment is maintained below 25° C.

This solution is allowed to separate into two layers and the aqueous layer is extracted again with toluene.

Then the organic phases are joined and washed with 175 ml of water. Thereafter the toluene is dried with sodium sulphate and decolorized with 0.6 g of charcoal. After 1 h and 15 minutes, the solution is filtered. The solvent is evaporated under reduced pressure to dryness to give 17.45 g of solid residue. The HPLC purity is 98.48%.

21 ml of ethyl acetate is added to the residue, heated to reflux temperature and then cooled to 5° C. and stirred at the same temperature for one hour and filtered.

The resulting crystals are washed with 2 ml of ethyl acetate to give 16.87 g of wet crude anhydrous mirtazapine. The HPLC purity is 99.20%

The crude mirtazapine is crystallized with 15 ml of ethyl acetate. Thereafter the mixture is cooled to 0-5° C., and stirred at the same temperature for 1 hour.

After filtering and washing with 2 ml of ethyl acetate, the wet crystals are dried under reduced pressure at 70° C. until constant weight, to give 15.05 g (molar yield: 80.35% ) of purified anhydrous mirtazapine crystals. The HPLC purity is 99.58%. Residual solvents (as determined by gas chromatography): toluene below detection limit of 100 ppm, ethyl acetate 258 ppm.

### EXAMPLE 4

This example shows the preparation of mirtazapine using 1 part compound of formula (II)+1 part water+2.76 parts dehydrating agent (parts are in weight) and keeping the reaction mixture for 1 h at a temperature in the range from 60° to 100° C.

To a suspension of 20 g of 1-(3-hydroxymethylpyridin-2-yl)4-methyl-2-phenylpiperazine in 20 ml of deionized water, 30 ml of sulfuric acid (96.10 wt %) is added dropwise at 20 to 58° C. during 15 minutes. Then the solution is gradually heated to 100° C. in 1 hour.

As a result, a solution is obtained which is cooled to 22° C. and 30 ml of water are slowly added. Next 188 ml of toluene are added and then the pH of the mixture is adjusted to about 9 with 28% ammonium hydroxide. The temperature during the pH adjustment is maintained below 25° C.

This solution is allowed to separate into two layers and the aqueous layer is extracted again with toluene.

Then the organic phases are joined and washed with 175 ml of water. Thereafter the toluene is dried with sodium sulfate and decolorized with 0.6 g of charcoal. After 1 h and 15 minutes, the solution is filtered. The solvent is evaporated under reduced pressure to dryness to give 16.39 g of solid residue.

21 ml of ethyl acetate is added to the residue, heated to reflux temperature and then cooled to 5° C. and stirred at the same temperature for one hour and filtered.

The resulting crystals are washed with 2 ml of ethyl acetate to give 13.68 g of wet crude anhydrous mirtazapine. The HPLC purity is 97.32%.

The crude is crystallized with 15 ml of ethyl acetate. Thereafter the mixture is cooled to 0-5° C. and stirred at the same temperature for 1 hour.

After filtering and washing with 2 ml of ethyl acetate, the wet crystals are dried under reduced pressure at 60° C. until constant weight, to give 11.18 g (molar yield: 59.09%) of purified anhydrous mirtazapine. The HPLC purity is 99.40%. Residual solvents (as determined by gas chromatography): toluene below detection limit of 100 ppm, ethyl acetate 321 ppm.

### EXAMPLE 5

This example shows the preparation of mirtazapine using 1 part compound of formula (II)+1 part water+2.76 parts dehydrating agent (parts are in weight) and keeping the reaction mixture for 3 h 10 min at 20° C.+1 h 30 min at 75-80° C.

To a suspension of 20 g of 1-(3-hydroxymethylpyridin-2-yl)-4-methyl-2-phenylpiperazine in 20 ml of deionized water, 30 ml of sulfuric acid (96.10 wt %) is added dropwise at 20 to 58° C. during 25 minutes. Then, the solution is stirred at room temperature for 3 hours. and 10 minutes. Next the solution is heated to 75-80° C. for 1 h 30 min.

As a result, a solution is obtained which is cooled to room temperature and 35 ml of water are slowly added. Next 188 ml of toluene are added and then the pH of the mixture is adjusted to about 9 with 28% ammonium hydroxide. The temperature during the pH adjustment is maintained below 25° C.

This solution is allowed to separate into two layers and the aqueous layer is extracted again with toluene.

Then the organic phases are joined and washed with 175 ml of water. Thereafter the toluene is dried with sodium sulfate and decolorized with 0.6 g of charcoal. After 1 h and 15 minutes, the solution is filtered. The solvent is evaporated under reduced pressure to dryness to give 16.65 g of solid residue. The HPLC purity is 98.26%.

21 ml of ethyl acetate is added to the residue, heated to reflux temperature and then cooled to 5° C. and stirred at the same temperature for one hour and filtered.

The resulting crystals are washed with 2 ml of ethyl acetate to give 14.49 g of wet crude anhydrous mirtazapine. The HPLC purity is 99.70%.

The crude mirtazapine is crystallized with 18 ml of ethyl acetate. Thereafter the mixture is cooled to 0-5° C. and stirred at the same temperature for 1 hour.

After filtering and washing with 2 ml of ethyl acetate, the wet crystals are dried under reduced pressure at 60° C. until constant weight, to give 12.1 g (molar yield: 64.60%) of purified anhydrous mirtazapine. The HPLC purity is 99.96%. Residual solvents (as determined by gas chromatography): toluene below detection limit of 100 ppm, ethyl acetate 229 ppm.

### EXAMPLE 6

This example shows the preparation of mirtazapine using 1 part compound of formula (II)+4.64 parts dichloromethane+4.6 parts dehydrating agent (parts are in weight) and keeping the reaction mixture for 16 h at room temperature.

10 g of 1-(3-hydroxymethylpyridin-2-yl)-4-methyl-2-phenylpiperazine are suspended in 35 ml of dichloromethane, then 25 ml of sulfuric acid (98.08 wt %) is added in 10 minutes. The mixture is maintained at room temperature for 16 hours.

Maintaining internal temperature below 25° C., 50 ml of water are added. Next 25 ml of toluene are added and the mixture is cooled. Then the pH of the mixture is adjusted to about 9 with 30% ammonium hydroxide. At this time, 50 ml more of toluene are added and the solution is allowed to separate into two layers and the aqueous layer is extracted again with toluene.

Then the organic phases are joined and the toluene is dried with sodium sulfate and filtered. The solvent is evaporated under reduced pressure to dryness to give 8.76 g of a solid residue. The HPLC purity of this residue of mirtazapine is 96.99%.

8 g of the obtained residue are dissolved in 9 ml of hot ethyl acetate and then cooled to 5° C. and stirred at the same temperature for one hour and filtered to give 7.05 g of wet crude anhydrous mirtazapine. The HPLC purity is 98.70%.

The crude mirtazapine is crystallized with 5 ml of ethyl acetate. Thereafter the mixture is cooled to 0-5° C. and stirred at the same temperature for 1 hour. After filtering, the wet crystals are dried under reduced pressure at 60° C. until constant weight, to give 6.4 g (molar yield: 74.98%) of purified anhydrous mirtazapine. The HPLC purity is 99.73%. Residual solvents (as determined by gas chromatography): toluene below detection limit of 100 ppm, dichloromethane below detection limit of 100 ppm, ethyl acetate 154 ppm.

1. A process for preparing mirtazapine comprising the steps of
(a) dissolving or suspending a compound of the formula (II) 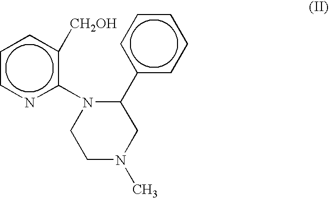
in a liquid diluent to obtain a first mixture;
(b) adding a ring closing reagent to the first mixture to obtain a second mixture;
(c) allowing the formation of mirtazapine in the second mixture;
(d) isolating mirtazapine from the second mixture.

2. The process of claim 1, wherein the liquid diluent is selected from the group consisting of halogenated hydrocarbons, hydrocarbons and water.

3. The process of claim 2, wherein the liquid diluent is selected among dichloromethane and water.

4. The process of claim 1, wherein the ring closing reagent is selected from the group consisting of sulfuric acid, concentrated sulfuric acid, phosphoric acid and phosphorous oxychloride.

5. The process of claim 4, wherein the ring closing reagent is

6. The process of claim 1, wherein the step of allowing the formation of mirtazapine comprises keeping the second mixture at a temperature below 90° C. for a time of 1 to 4 hours.

7. The process of claim 1, wherein the step of isolating mirtazapine involves contacting the second mixture with water or with an aqueous solution to obtain a third mixture comprising an aqueous phase.

8. The process of claim 7, wherein the pH of the aqueous phase is adjusted to not less than 8.

9. The process of claim 7, wherein the step of isolating mirtazapine involves the addition of a non-water miscible solvent.

10. The process of claim 9, wherein the non-water miscible solvent is selected from the group consisting of toluene and dichloromethane.

11. The process of claim 9, wherein the step of isolating mirtazapine involves the separation of the non-water miscible phase containing mirtazapine from the aqueous phase.

12. The process of claim 11, wherein the aqueous phase is extracted one or more times with a non-water miscible solvent to obtain an additional portion of non-water miscible phase.

13. The process according to claim 11, wherein the non-water miscible phase is subjected to decolorization.

14. The process of claim 9, wherein the step of isolating mirtazapine involves in addition removing the solvent.

15. The process of claim 9, comprising the additional step of purifying the isolated mirtazapine by crystallizing it one or more times from an organic ester solvent.

16. The process of claim 15, wherein the solvent is ethyl acetate.

17. The process of claim 15, wherein the mirtazapine is obtained in anhydrous crystalline form.

18. The process of claim 1 wherein
(a) the compound of the formula (II) is mixed with water to obtain the first mixture;
(b) sulfuric acid is added as a ring closing reagent to obtain the second mixture;
(c) the formation of mirtazapine in the second mixture is allowed keeping the second mixture at a temperature below 90° C. for a time of 2 hours;
(d) mirtazapine is isolated from the second mixture by contacting with water, addition of toluene adjustment of the pH of the aqueous phase to 8 to 10, subsequent separation of the toluene phase, decolorization and then removal of the toluene by evaporation.

19. The process of claim 18, comprising the additional step of purifying the obtained mirtazapine by crystallization from ethyl acetate.
